(19) United States
(12) Patent Application Publication
(10) Pub. No.: US 2025/0025072 A1
(43) Pub. Date: Jan. 23, 2025

(54) DEVICE AND METHOD FOR MEASURING BLOOD CONSTITUENTS

(71) Applicant: Samsung Electronics Co., Ltd., Suwon-si (KR)

(72) Inventors: Vladislav Valerievich LYCHAGOV, Moscow (RU); Anton Sergeevich MEDVEDEV, Moscow (RU); Elena Konstantinovna VOLKOVA, Moscow (RU); Gennady Dmitrievich MAMYKIN, Moscow (RU)

(21) Appl. No.: 18/909,345

(22) Filed: Oct. 8, 2024

Related U.S. Application Data
(63) Continuation of application No. PCT/KR2022/014353, filed on Sep. 26, 2022.

(30) Foreign Application Priority Data
May 19, 2022 (RU) 2022113451

(51) Int. Cl. A61B 5/1455; A61B 5/00; G16H 10/40; G16H 50/70

(52) U.S. Cl. CPC A61B 5/1455; A61B 5/7225; G16H 10/40; G16H 50/70; A61B 2562/0238

(57) ABSTRACT

A method for measuring blood constituents is provided. The method includes radiating an area of a subject's body, containing blood constituents and dynamic and static tissue components, with at least two light beams, detecting at least one backscattered light feedback from blood constituents and dynamic and static tissue components, the feedback representing at least one interferometric signal from each of the at least two light sources, filtering the at least one interferometric signals in accordance with a frequency band which contains frequencies of interferometric oscillations, defining dynamics of blood constituents, performing analog-to-digital (AD) conversion of the filtered signals at a frequency corresponding to the frequency band, to form discrete filtered signals, obtaining from the discrete filtered signals, a set of spectral-time features of signals, and obtaining concentration of the blood constituents based on functional relationships of the obtained set of spectral-time features of signals and concentrations of the blood constituents.

### CROSS-REFERENCE TO RELATED APPLICATION(S)

This application is a continuation application, claiming priority under § 365(c), of an International application No. PCT/KR2022/014353, filed on Sep. 26, 2022, which is based on and claims the benefit of a Russian patent application number 2022113451, filed on May 19, 2022, in the Russian Intellectual Property Office, the disclosure of which is incorporated by reference herein in its entirety.

### BACKGROUND

### 1. Field

The disclosure relates to devices and methods for measuring blood constituents. More particularly, the disclosure relates to means for noninvasive, personal and/or on-demand health monitoring, in particular, monitoring and measurement of blood parameters. These devices can be used in wearable measuring systems, wrist watches, smart watches, stationary diagnostic devices, consumer devices and gadgets for personal healthcare.

### 2. Description of Relate Art

With the growing popularity of wearable measuring systems for monitoring health of a subject, the demand is increasing for compact means for noninvasive, personal, and/or on-demand health monitoring, in particular, for monitoring measurements of concentrations of various forms of hemoglobin Hb: oxygenated Hb, de-oxygenated Hb, met-Hb, carboxy-Hb, and other properties of blood.

The main requirement for these systems is high accuracy of measurements, high sensitivity to changes in measured parameters, low sensitivity to changes in measurement conditions (motion artefacts), with simultaneous requirement of compactness and the ability for taking measurements in real time.

Therefore, the object of the disclosure, from the viewpoint of design of the device for measuring blood constituents, is to provide a compact wearable device capable of providing increased spectral resolution and, accordingly, high accuracy of measurements, which is robust and resistant to any motions in measurement process, for use in wearable consumer gadgets and similar devices.

FIG. 1A is an optical diagram of a conventional system for measuring blood constituents, where light beams pass from broadband light-emitting diodes (LEDs) through components of subject's studied area and are registered at a photodetector and a schematic view of obtaining total light intensity at the photodetector diagram of FIG. 1A according to the related art.

FIG. 1B shows plots of variations in signal 1 for LED₁ and signal 2 for LED₂ when passing through the analyzed subject's area, and signal 1 and signal 2 at the photodetector (PD) of the conventional system for measuring blood constituents of FIG. 1A according to the related art.

Solutions of such type are based on the following principles described with reference to FIGS. 1A and 1B, according to the related art.

Target area of a subject is radiated with several light sources, in this case with broadband LEDs: LED₁ and LED₂ each having own wavelength range: LED₁ corresponds to Δλ₁, and LED₂ corresponds to Δλ₂.

Furthermore, the studied components of the subject's area to be monitored are conditionally divided into target components including erythrocytes, blood, and non-target components including other components, tissue, chromophores, etc. Upon passing through the target area, and, respectively, through the target and non-target components (see FIG. 1A), the light beams from different LEDs undergo changes.

In these conditions, light interacts with both target and non-target tissue components, in particular, it is partially absorbed and partially scattered. Light at different wavelengths interacts differently with target and non-target tissue components. For example, a target component may predominantly absorb one wavelength, while a non-target component absorbs a different wavelength. At the same time, due to the fact that spectra of broadband light sources (LEDs) may partially overlap, the resulting intensity from different LEDs may coincide, despite the fact that light from these light sources was absorbed by different tissue components.

Thus, when passing through the subject's target area, light beams output from different LEDs enter photodetector (PD), which measures total intensity of light from different LEDs after passing through the target and non-target components of the subject area under study, see FIG. 1A. It should be noted that during measurements of the subject's studied area, any movement of both the subject and the device gives rise to appearance of artefacts, such as interference and noise. Plots in FIG. 1B show behavior of signal 1 for LED₁ and signal 2 for LED₂, passing through the subject's studied area, and signal 1 and signal 2 at photodetector (PD), where signal 1 signal 2.

In general, light from LED₁ and LED₂ may partially overlap. Emission from both sources contains common spectral components, indicated in FIG. 1A as “--->” and “-.-.-.>”. In this case, light of LED₁ is predominantly absorbed by one tissue component (e.g. target component), and light of LED₂ is predominantly absorbed by another tissue component (e.g. non-target component). Light emitted from LED₁ contains spectral components that can be absorbed by non-target component, and Light emitted from LED₂ contains spectral components that can be absorbed by target tissue component. Despite different ratio of spectral components in the light passing through the tissue sample, the photodetector cannot distinguish between them, and the photodetector output from both LEDs will be approximately the same (signal 1≈signal 2). Thus, broadband light source cannot provide sufficient spectral resolution to spectrally separate signals from different tissue components.

This conventional design precludes direct detection of signal from a target component of the subject area under study, since signal from other non-target components is mixed with signal from target constituents, erythrocytes, blood, so only total intensity of signals from different components of the subject area under study can be measured at the photodetector, and measurements of blood parameters are inaccurate.

Tissue blood supply processes are associated with a pulse wave, i.e. change in the amount of blood in the measured tissue volume and, accordingly, blood signal in this case is characterized by a weak variable component (AC) against the background of a strong constant component (DC).

In general, determination of blood parameters and constituents using photoplethysmography (PPG) is based on measurement of variable component (AC). Correct measurement of variable component (AC) of photoplethysmographic signal requires accumulation of multiple pulse wave cycles, so the process of measurement or data acquisition usually takes several seconds. However, living tissue or living organism of a subject may change in a few seconds of measurements, i.e. it is unstable, and the effect of any movements occurring at photosensor or other elements of the measurement device gives rise to unwanted artefacts or noise, significantly impairing the accuracy of measurements of blood constituents.

The studies of conventional systems for measuring blood constituents allow for the conclusion that these systems suffer from the following basic drawbacks:
1) low measurement accuracy due to low spectral resolution,
2) motion artefacts that occur when measuring blood,
3) high power consumption due to long data acquisition time.

The disclosure is aimed to overcome all of the above drawbacks inherent in conventional devices for measuring blood constituents.

Hereinafter, similar conventional solutions for measuring blood constituents are described.

Patent application US20190374140A1, publ. 12.12.2019, IPC A61B5/00, discloses a system and method for determining peripheral oxygen saturation (SpO₂) and hemoglobin concentration using multi-spectral laser imaging (MSLI). The system consists of at least two different light sources with different wavelengths, a camera configured to simultaneously receive information related to the first and second light sources from the sample and a processor to form the synthesized image. The drawback of this system (MSLI) is the need to include additional visualization tools in it, in this case a camera for forming a two-dimensional image.

Further, patent application US20180214025A1, publ. 02.08.2018, IPC A61B5/00, discloses a systems and method for detecting the flow of blood or other fluids in biological tissue by illuminating the biological tissue with two or more beams of coherent light and detecting responsively emitted light. Dynamic parameters of blood are determined based on time and/or spatially varying light interference pattern obtained from the biological tissue (e.g. speckle patterns), or some other properties of the detected light. Drawbacks of the system and method include specific requirements on electronics and processing units to measure absolute parameters of blood flow, speed, etc.

Another prior art is a stationary physiological measurement system, disclosed in patent application U.S. Ser. No. 10/123,726B2, publ. 13.11.2018, IPC A61B 5/14552, the system is based on a typical LED pulse oximeter and comprises a sensor, a processor, a communications link and information elements. The sensor is configured to transmit light having a plurality of wavelengths into a tissue site and to generate a sensor signal responsive to the transmitted light after tissue attenuation. The processor is configured to operate on the sensor signal so as to derive at least one physiological parameter. The disadvantage of this system, in contrast to the present wearable measurement device, is that it cannot be used in mobile apparatuses and gadgets.

The closest prior art to the disclosure is disclosed in International Publication WO2016178986A1, publ. 10.11.2016, IPC A61B5/1455, which discloses a system and method for SpO₂ determination using reflective PPG. The measurement method is applied to physiological signal analysis, and the measurement system facilitates SpO₂ prediction by basing a calibration step on the ratio of red and infrared path lengths. The system facilitates SPO₂ prediction, on any user for a given optical configuration, by ensuring that path length is accordingly incorporated into the prediction equation. In addition, use of an automatic gain control implemented on the system enables selection of optimal signal for determining SpO₂. The system relates to typical pulse oximetry system operating in reflective mode, susceptible to motion artefacts, and having low accuracy and sensitivity because of wideband light sources used.

Therefore, conventional means for measuring blood parameters do not satisfy the requirements imposed on them by consumers. At the same time, the demand has grown tremendously for wearable systems for measuring blood constituents, and in particular, systems for noninvasive measurement of concentrations of various forms of hemoglobin Hb: oxygenated Hb, de-oxygenated Hb, met-Hb, carboxy-Hb and blood oxygen concentration.

Moreover, hemoglobin values and blood oxygen levels (saturation) are relevant for identifying the condition of subjects, and any deviation from typical ranges for population groups indicates a potential risk of diseases depending on hemoglobin concentration and/or blood oxygen levels. There are many blood measurement systems on market that measure blood constituents, but many of them are not suitable for continuous monitoring of blood parameters, require blood sampling, need an additional device (heart rate monitor), which is often inconvenient for measurements outside of medical institutions. Consequently, there is a need for a noninvasive and non-intrusive method for measuring and monitoring concentration of hemoglobin in blood and oxygen content in blood, while enabling monitoring of blood parameters over a period of time (several seconds) without a large amount of tests, rather than a one-time measurement of blood parameters. The inventors have carried out studies of similar conventional systems for measuring blood parameters on the market and identified the following characteristics of conventional systems in comparison with the disclosure.

The systems were analyzed by the following characteristics:

use of a separate measurement device, accuracy of parameter measurements, power consumption, continuous monitoring, motion instability.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a device and method for measuring blood constituents.

Additional aspects will be set forth in part in the description which follows and, in part, will be apparent from the description, or may be learned by practice of the presented embodiments.

In accordance with an aspect of the disclosure, a method for measuring blood constituents is provided. The method includes radiating an area of a subject's body, containing blood constituents and dynamic and static tissue components, with at least two light beams using at least two light sources, detecting, using at least one photodetector, at least one backscattered light feedback from blood constituents and dynamic and static tissue components, wherein the feedback represents at least one interferometric signal from each of the at least two light sources, filtering, using a filter, the at least one interferometric signal in accordance with a signal frequency band that contains frequencies of interferometric oscillations, performing analog-to-digital (AD) conversion of the filtered signals at a frequency corresponding to the given frequency band of the filtered signal, to form discrete filtered signals, obtaining from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals, defining spectral and dynamic properties of the blood constituents, where λ1, λ2, λ3, . . . , λn are the wavelength indexes. t1, . . . tk are the time feature indexes, and where the spectral-time feature v_{λ}_{ₙ}^{tk} corresponds to time feature tk measured at wavelength λn, and obtaining a concentration of the blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression: c₁≈ƒ₁(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), c₂≈ƒ₂(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), . . . , cₘ≤ƒₘ(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), where c1 . . . cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

In accordance with another aspect of the present disclosure, a device for measuring blood constituents is provided. The device includes at least two light sources, at least one photodetector, a filter, an analog-to-digital (AD) converter, memory storing one or more computer programs, and one or more processors communicatively coupled to the at least two light sources, the at least one photodetector, the filter, the AD converter, and the memory, wherein the one or more computer programs include computer-executable instructions that, when executed by the one or more processors individually or collectively, cause the device to radiate an area of a subject's body, containing blood constituents and dynamic and static tissue components, with at least two light beams using at least two light sources, detect, using the at least one photodetector, at least one backscattered light feedback from the blood constituents and dynamic and static tissue components, wherein the feedback represents at least one interferometric signal from each of the at least two light sources, filter, using the filter, the at least one interferometric signal in accordance with a signal frequency band which contains frequencies of interferometric oscillations, perform analog-to-digital (AD) conversion of the filtered signals at a frequency corresponding to the signal frequency band of the filtered signal, to form discrete filtered signals, obtain, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), defining spectral and dynamic properties of the blood constituents, where λ1, λ2, λ3, . . . , λn are wavelength indexes, and where t1, . . . tk are time feature indexes, with spectral-time feature v_{λ}_{ₙ}^{tk} corresponding to time feature tk measured at wavelength λn, and obtain a concentration of the blood constituents based on the obtained set of spectral-time features(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression: c₁≈ƒ₁(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) c₂≈ƒ₂(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) cₘ≈ƒₘ(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), where c1, c2, . . . ,cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

In accordance with another aspect of the present disclosure, one or more non-transitory computer-readable storage media storing one or more computer programs including computer-executable instructions that, when executed by one or more processors of an electronic device individually or collectively, cause the electronic device to perform operations are provided. The operations include radiating an area of a subject's body, containing blood constituents and dynamic and static tissue components, with at least two light beams using at least two light sources, detecting, using at least one photodetector, at least one backscattered light feedback from the blood constituents and dynamic and static tissue components, wherein the feedback represents at least one interferometric signal from each of the at least two light sources, filtering, using a filter, the at least one interferometric signal in accordance with a signal frequency band which contains frequencies of interferometric oscillations, performing analog-to-digital (AD) conversion of the filtered signals at a frequency corresponding to the signal frequency band of the filtered signal, to form discrete filtered signals, obtaining, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), defining spectral and dynamic properties of the blood constituents, where λ1, λ2, λ3, . . . , λn are wavelength indexes, and where t1, . . . tk are time feature indexes, with spectral-time feature v_{λ}_{ₙ}^{tk} corresponding to time feature tk measured at wavelength λn, and obtaining a concentration of the blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression: c₁≈ƒ₁(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) c₂≈ƒ₂(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) . . . cₘ≈ƒₘ(v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), where c1, c2, . . . ,cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), and where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of the certain embodiments of the disclosure will be more apparent from the following description taken in conjunction with the accompanying drawings, in which:

FIG. 1A is an optical diagram of a conventional system for measuring blood constituents, where light beams pass from broadband light-emitting diodes (LEDs) through components of subject's studied area and are registered at a photodetector and a schematic view of obtaining total light intensity at the photodetector diagram of FIG. 1A according to the related art;

FIG. 1B shows plots of variations in signal 1 for LED₁ and signal 2 for LED₂ when passing through the analyzed subject's area, and signal 1 and signal 2 at the photodetector (PD) of the conventional system for measuring blood constituents of FIG. 1A according to the related art;

FIG. 2A shows plots of broadband and narrowband emission spectra, where two narrowband light sources can emit at wavelengths sufficiently separated from each other and coinciding with absorption wavelengths of non-target and target components, but not coinciding with each other; the broadband light source has a sufficiently wide spectrum including absorption wavelengths of both target and non-target components according to an embodiment of the disclosure;

FIG. 2B shows plots of several broadband and narrowband light sources, central wavelengths of which coincide with absorption wavelengths of different target components, where even if central wavelengths of broadband source light exactly coincide with absorption bands of desired components, the other part of emission spectrum of these sources coincides with another component according to an embodiment of the disclosure;

FIG. 2C shows plots of absorption coefficient μₐ(λ) of blood constituents versus wavelength λ (nm) according to an embodiment of the disclosure;

FIG. 2D is a schematic diagram illustrating a process of spectral selection occurring in analyzed tissue volume when radiated with two narrowband coherent light sources LD₁ with wavelength λ₁ and LD₂ with wavelength λ₂ according to an embodiment of the disclosure;

FIG. 2E is a schematic diagram illustrating the result of spectral selection occurring in the analyzed tissue volume according to FIG. 2D when selecting the target components according to an embodiment of the disclosure;

FIG. 3A shows amplitudes of a constant (slowly changing) signal component and a fluctuating (rapidly changing) signal component according to an embodiment of the disclosure;

FIG. 3B shows curves defining signal power (P) versus signal frequency (F), where solid line stands for power of original signal containing both slowly changing and rapidly changing components, and dotted line stands for power of fluctuating component only according to an embodiment of the disclosure;

FIG. 3C shows amplitude of resulting signal as a function of time after appropriate processing, which keeps only fluctuating component of the signal according to an embodiment of the disclosure;

FIG. 4A is a schematic diagram of a process of selection by dynamic properties of components, which occurs in the analyzed tissue volume when radiated with narrowband coherent light source LD₁ with wavelength λ₁ according to an embodiment of the disclosure;

FIG. 4B is a schematic diagram illustrating the result of selecting components by dynamic properties, occurring in the analyzed tissue volume of FIG. 4A when selecting target components according to an embodiment of the disclosure;

FIG. 4C is the amplitude of signal fluctuating as a result of interference of light scattered on moving component K-1 and reference light according to an embodiment of the disclosure;

FIG. 4D is the amplitude of signal fluctuating as a result of mutual interference of light scattered on target moving component K-1 and target moving component K-2 according to an embodiment of the disclosure;

FIG. 4E is the amplitude of non-fluctuating (slowly varying) signal from static component K-A according to an embodiment of the disclosure;

FIG. 4F is a curve defining power of signal registered at photodetector (PD) as a function of frequency, in signal frequency band 0 corresponding to signal in FIG. 4E, in signal frequency band 1 corresponding to signal in FIG. 4D and signal frequency band 2 corresponding to signal in FIG. 4C according to an embodiment of the disclosure;

FIG. 5 is a schematic diagram illustrating a process occurring in analyzed tissue volume when radiated with multiple coherent light sources according to an embodiment of the disclosure;

FIG. 6 is a structural diagram of a wearable device for noninvasive measurements of blood constituents according to an embodiment of the disclosure;

FIG. 7A schematically shows variations in emission spectrum of a broadband light source (bold line) at wavelength 670 nm when passing through the analyzed volume, which is defined by transmission spectrum ((T(o)) according to an embodiment of the disclosure;

FIG. 7B schematically shows variations in emission spectrum of a broadband light source (bold line) at wavelength 805 nm when passing through the analyzed volume, which is defined by transmission spectrum ((T(o)) according to an embodiment of the disclosure;

FIG. 8A shows plots of signal curves for broadband light source (LED) and narrowband light source (laser diode, LD) versus total hemoglobin concentration (g/l) according to an embodiment of the disclosure;

FIG. 8B show plots of signal curves for broadband light source (LED) and narrowband light source (laser diode, LD), versus oxygenation level (%) according to an embodiment of the disclosure; and

FIG. 9 is a schematic diagram of noninvasive monitoring of hemoglobin concentration according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

In the context of the disclosure, the following concepts and terms shall have the following meanings assigned to them by the inventors:

Analyzed volume is the area of biological tissue of a subject (human, animal, etc.) to be studied.

Sample is an object to be studied or a section of subject's biological tissue (tissue, blood, etc.)

Studied blood constituent is a target component of biological tissue to be studied.

Target component is a component of biological tissue to be studied, which absorbs light at wavelengths λ₁ . . . λₙ and is defined by movement and/or pulsation during blood flow.

Non-target component is a component of biological tissue to be studied, which absorbs light at wavelengths λ₁ . . . λₙ a or other wavelengths and is defined by absence of movement.

Motion artefacts include interference, noise, periodic or non-periodic, random, or having some regularity, signal components having parameters that are not characteristic of a pulse wave and are caused by any mechanical displacement of measuring apparatus and measured object relative to each other.

PPG (photoplethysmography) measurement—is a method of spectrophotometric measurement of the degree of absorption of light at various wavelengths in order to qualitatively and quantitatively analyze composition of an object, in particular biological tissue.

Interferometric signals are signals obtained as a result of interference of two parts of light, for example, reference light, which can be either light scattered by a moving particle of the analyzed volume, or any other external light, or light scattered by a static particle of the analyzed volume.

Frequency of interferometric oscillations is the frequency at which interference signal fluctuates, which represents difference frequency (period). Difference frequency (period) of interference signal is proportional to difference between parameters, for example, frequency, wavelength of two interfering parts of light, i.e. reference light, which can be either light scattered by a moving particle of the analyzed volume, or any other external light, or light scattered by a static particle of the analyzed volume.

The disclosure provides a device and method for noninvasive measurements of blood constituents, and can be used in wearable measuring devices, wrist watches, smart watches, stationary diagnostic devices, consumer devices and gadgets for personal healthcare.

Basic objectives of the disclosure are:
to increase spectral resolution of measurements being taken;
eliminate influence of movements of measuring device and measured object relative to each other;
provide selective sensitivity of measuring apparatus to dynamic components of tissue (blood);
reduce power consumption of measuring apparatus.

The objectives are attained by the provision of a wearable and method for measuring of blood constituents, which ensure more accurate measurements of blood constituents, while reducing the device power consumption.

Embodiments of a device and method for measuring blood constituents are described below with reference to the accompanying drawings. It is worthwhile to turn attention to some fundamental issues of measurements disclosed in the present description.

As is well known, blood is a liquid and mobile connective tissue of the body internal environment, which consists of liquid medium in the form of plasma and suspended therein formed elements (cells and cell derivatives): erythrocytes, leukocytes and thrombocytes, etc. Blood circulates through a closed system of vessels under the force of rhythmically contracting heart, and the speed of blood flow and its pulsation vary depending on the type of vessels through which it flows. In larger vessels, arterioles, blood flows more quickly and is pulsating, while in smaller vessels, capillaries, blood flows more slowly and there is little or no pulsation.

Depending on the absorption ability of various blood constituents under light radiation, the components can be conditionally classified by spectral properties and by dynamic properties, for example, by the speed of blood flow and its pulsation.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

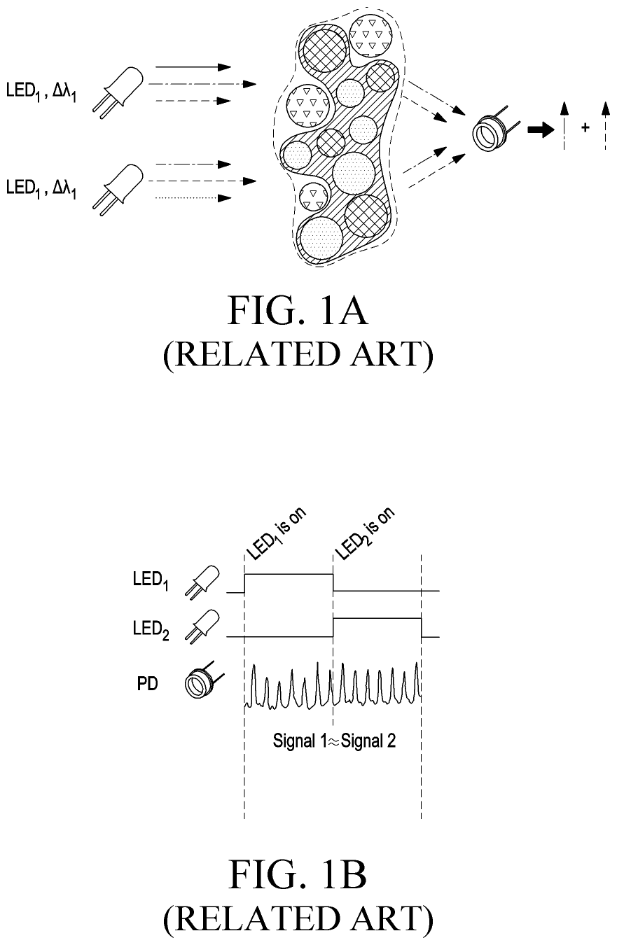

As already mentioned above, when the analyzed volume is radiated by several light sources, as an example, see FIG. 1A, LED₁ and LED₂ each having own wavelength range: Δλ₁ for LED₁ and Δλ₂ for LED₂. Light beams interact with blood constituents and other tissue components of the analyzed volume and are detected by photodetector PD.

Furthermore, various blood constituents and tissues differently absorb light at different wavelengths.

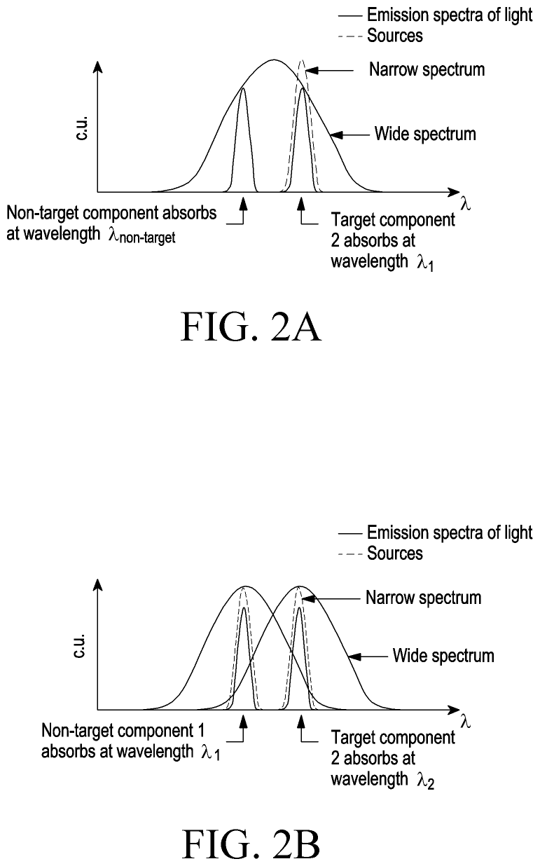

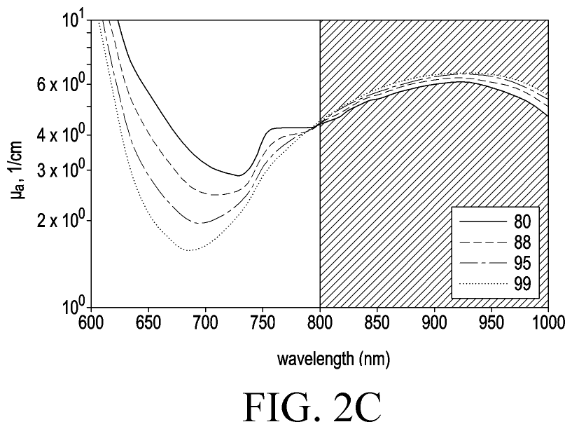

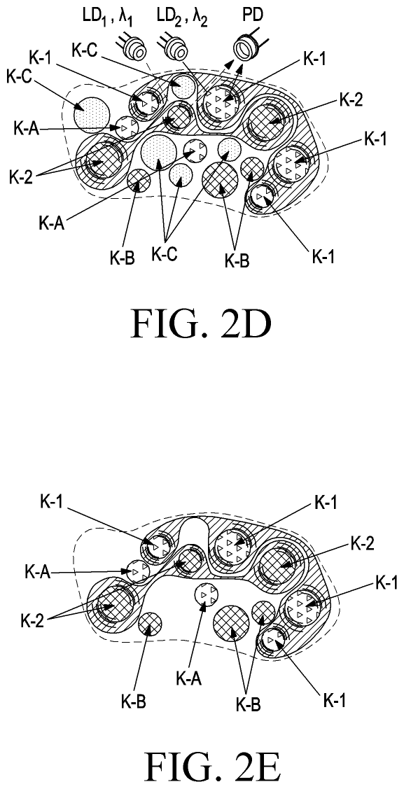

FIG. 2D is a schematic diagram illustrating a process of spectral selection occurring in analyzed tissue volume when radiated with two narrowband coherent light sources LD₁ with wavelength λ₁ and LD₂ with wavelength λ₂ according to an embodiment of the disclosure.

FIG. 2E is a schematic diagram illustrating the result of spectral selection occurring in the analyzed tissue volume according to FIG. 2D when selecting the target components according to an embodiment of the disclosure.

Referring to FIG. 2D for clarity, various fractions or components that make up target components (for example, component 1 (K-1) and component 2 (K-2)) absorb light at wavelengths Δλ₁ and Δλ₂ and are defined by pulsation in the blood flow, while the others, non-target components (K-A) and (K-B), including tissue, chromophores, may or may not absorb light at wavelengths Δλ₁ and Δλ₂, but are not defined by pulsation or movement in the blood flow.

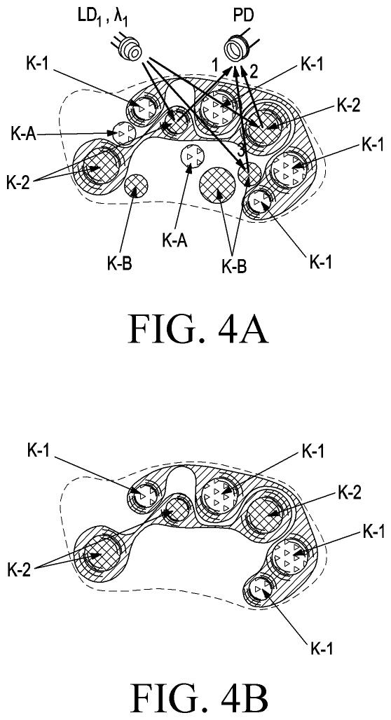

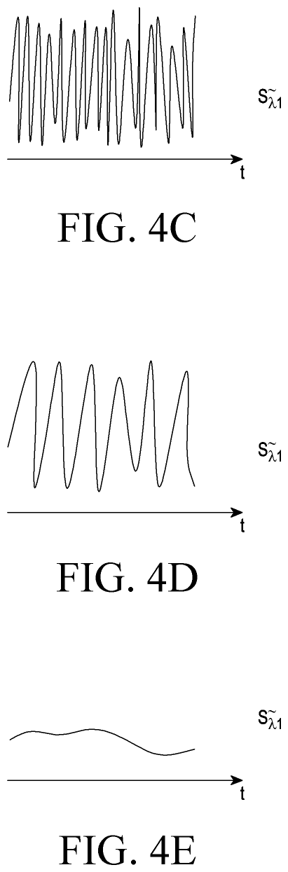

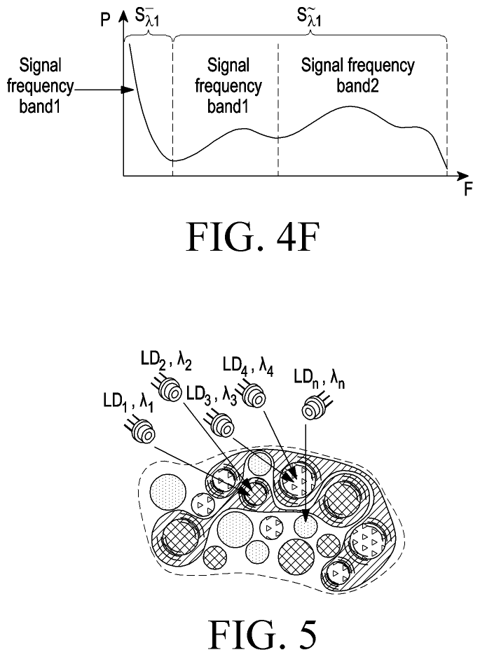

Thus, to separate and independently measure target blood constituents (see FIGS. 2D, 2E, 4A, and 4B), two conditions are to be met: the components should selectively absorb at a certain wavelength and move and/or pulsate.

Next, key points of the disclosure will be disclosed.

### Key Point 1:

### Step 1

Selection of blood constituents by their spectral properties. In the measurement process, only those blood constituents are selected that absorb light emitted by a coherent light source at predetermined wavelengths, for example, λ₁, and λ₂.

In this case, selectivity by spectral properties is ensured by high spectral resolution due to the use of a coherent light source.

### Key Point 2:

### Step 2

Selection of blood constituents by their dynamic properties. In the measurement process, only those components (biological tissue) are selected that are moving and/or pulsating. Sensitivity to movement is ensured by the use of a coherent light source.

It should be noted that step 1 and step 2 are not separated in time, and occur simultaneously, particularly, spectral selection and detection of an interferometric signal at a certain wavelength are executed simultaneously.

### Step 3

Direct measurement of concentration of the selected blood constituents parameters, selected in steps 1 and 2, conventionally component K-1 and component K-2 (see FIG. 2E).

### Step 4

Evaluation of measurement results by the user of the measurement device. As a result, the user is informed about normal or abnormal values of blood constituents.

As mentioned earlier, target components are selected in the disclosure by their spectral properties (Key Point 1) and by dynamic properties (Key Point 2). Narrowband coherent light sources (laser diodes (LD)) are used in the measurement device, which provides high spectral resolution for sensitive and accurate separation of target components by their spectral properties and enables detecting an interference signal for sensitive and accurate separation of target components by their dynamic properties.

Furthermore, performance of Key Point 1 implies the following possible initial conditions, see FIGS. 2D, and 2E:
target component K-1 and non-target component K-A both absorb light at wavelength λ₂,
target component K-2 and non-target component K-B both absorb light at wavelength λ₁,
non-target component C absorbs neither λ₁ nor λ₂.

Measurement of absorption for each blood constituent at wavelengths λ₁, λ₂, makes it possible to exclude non-target component K-C from the measurements (see FIG. 2E).

Signals arriving at photodetector PD after passing through the analyzed volume carry information about each of the blood constituents, for example, signal S_{λ1} is registered at the PD from light source LD₁, when it is initiated, and carries information about target component K-1 and non-target component K-A, and signal S_{λ2} is registered at the PD from light source LD₂, when it is initiated, and carries information about target component K-2 and non-target component K-B.

The disclosure uses predominantly narrowband light sources to measure target blood constituents, which ensures accuracy of the measurements taken. On the other hand, conventional solutions use broadband light sources and the measurement process is performed for both target blood constituents and non-target components, and as a result does not provide the required measurement accuracy.

Moreover, in the disclosure, when narrowband light sources are used, various blood constituents, for example, K-1 component and K-2 component, are measured separately, which eliminates overlapping the emission spectra.

FIGS. 2A and 2B show plots illustrating emission spectra of narrowband and broadband light sources, where solid line stands for broadband spectrum, and dotted line stands for narrowband spectrum according to various embodiments of the disclosure.

Referring to FIGS. 2A and 2B combine different parameters: emission spectra and absorption coefficients (i.e. curves defining emission spectra and curves defining absorption coefficients versus wavelength), for illustrative purposes only.

FIG. 2A shows plots of broadband and narrowband emission spectra, where two narrowband light sources can emit at wavelengths sufficiently separated from each other and coinciding with absorption wavelengths of the non-target and target components, but not coinciding with each other, and the broadband light source has a sufficiently wide spectrum to include absorption wavelengths of both target component (λ₁) and non-target component (λ_{non-target}).

FIG. 2B shows plots of several broadband and narrowband light sources whose central wavelengths coincide with absorption wavelengths of different target components (λ₁,λ₂); here, even if the central wavelengths of broadband sources exactly coincide with absorption bands of desired components, the other part of emission spectrum of these sources coincides with the other component.

Thus, when a broadband light source is used, it is essentially impossible to measure the degree of light absorption (or another spectral characteristic) at one specific wavelength and, therefore, it is impossible to selectively measure concentration (or any other parameter) of a single target component of tissue.

FIG. 2C shows plots of absorption coefficient μₐ(λ) of blood constituents versus wavelength λ (nm) according to an embodiment of the disclosure.

In addition, FIG. 2C shows plots of absorption coefficient μₐ(λ) in (1/cm) of blood constituents as a function of wavelength λ(nm), corresponding to blood oxygenation levels of 80%, 88%, 95% and 99%.

As clearly shown in plots of FIG. 2C, signals registered at the photodetector, which define absorption coefficients, can be different for different concentrations or levels of blood oxygenation and at different wavelengths, therefore, to obtain reliable measurement data, the signal must be measured at a precisely specified wavelength.

As mentioned earlier, one of advantages of the disclosure is high spectral resolution of the obtained measurement data, which ensures, as a result of data processing, accurate data on the components of subject's blood in real time. One of the factors providing this result is the use of narrowband coherent light sources.

In one of preferred embodiments of the disclosure, two light sources are used to radiate the analyzed tissue volume, a first one, LD₁, with wavelength λ₁, preferably in the visible wavelength range of <800 nm, and a second one, LD₂, with wavelength λ₂, preferably in the near infrared wavelengths range of >800 nm. For example, one laser diode may have wavelength λ₁=650 nm, and a second one-wavelength λ₂=940 nm. When silicon-based photodetectors are used, preferred ranges for selecting λ₁, and λ₂ are: 400 nm to 800 nm and 800 nm to 1100 nm, respectively. The disclosure does not exclude the use of other light sensors. In this case, the choice of wavelengths can be extended to the range of <400 nm and >1100 nm, respectively.

However, the above ranges are not limited to the values presented, but are given in the description only as an example.

Hereinafter, Key Point 1 of the disclosure will be described in detail.

As earlier mentioned in the disclosure, blood constituents are selected by their spectral properties. Thus, in the measurement process, only those blood constituents are selected that absorb light emitted by a coherent light source with predetermined wavelengths, for example λ₁, λ₂, . . . λₙ.

For further analysis, total hemoglobin (tHb) can be simplified as consisting of two components: oxygenated (O₂Hb) and non-oxygenated (RHb) hemoglobin. Without loss of generality of the conclusions, the contribution of other forms of hemoglobin, for example, methemoglobin (MetHb), sulfhemoglobin (SHb), to the absorption spectrum of whole blood can be assumed to be negligible.

Light absorption at different wavelengths by different blood constituents is determined by extinction coefficient (e(λ)).

In accordance with the above assumption, the extinction coefficient of whole blood will be determined by extinction coefficients of oxygenated and reduced hemoglobin in the following ratio:

$${e}_{tHb} ( λ ) = {e}_{O 2 Hb} ( λ ) \cdot S + {e}_{RHb} ( λ ) \cdot ( 1 - s ) \qquad Equation 4$$

where e_{tHb}(λ), e_{O2Hb}(λ), e_{RHb}(λ) are extinction coefficients of total hemoglobin, oxygenated hemoglobin and reduced hemoglobin depending on the wavelength λ, respectively,

S is saturation (S_{P}O₂), i.e. proportion of oxygenated hemoglobin (O₂Hb) relative to total hemoglobin in blood.

For calculations, values of extinction coefficients can be taken from the publication: Steven L Jacques, Optical properties of biological tissues: a review, Physics in Medicine & Biology, Volume 58, Number 11, 2013.

Light absorption (dimensionless value) by a tissue sample depends on concentration of analyzed component in the tissue (in this case, blood constituents), optical path from the light source to the photodetector, and wavelength, and is defined by equation 5:

$$A ( λ ) = \frac{e [ \frac{L}{mol \cdot cm} ] \cdot C [ \frac{g}{L} ] \cdot l [ cm ]}{MW [ \frac{g}{mol} ]} \qquad Equation 5$$

where A(λ) is absorption of tissue sample as a function of concentration c of analyzed blood constituent, in this case, hemoglobin, optical path length 1, wavelength λ are considered,
e is extinction coefficient of hemoglobin, e.g. total hemoglobin, oxygenated hemoglobin and reduced hemoglobin, depending on the wavelength λ,
c is concentration of hemoglobin,
MW(g/mol) is molecular weight of a hemoglobin,
l(cm) is length of optical path of light from light source, passed through the sample and incident on photodetector.

To visualize absorption spectra, it is convenient to operate on absorption coefficient μₐ (1/cm) using equation 6:

$${μ}_{a} ( λ ) ∼ \frac{e [ \frac{L}{mol \cdot cm} ] \cdot C [ \frac{g}{L} ]}{MW [ \frac{g}{mol} ]} \qquad Equation 6$$

e is extinction coefficient of hemoglobin, e.g. total hemoglobin, oxygenated hemoglobin and reduced hemoglobin, depending on wavelength λ,
c is concentration of hemoglobin,
MW(g/mol) is molecular weight of hemoglobin.

Transmission (T(X)) is ratio of the intensity of emission passed through a sample (component under study) to the intensity of emission incident on the sample and is related to absorption by the equation:

$$T ( λ ) = {10}^{- A ( λ )} \qquad Equation 7$$

where A(λ) is absorption of sample (blood constituents and/or tissues) depending on wavelength λ.

Now the influence of source emission spectrum width on the possibility of spectral separation of absorbing components in the analyzed tissue volume will be described in more detail. To do this, the process of light absorption for given spectral properties of the source and the analyzed tissue sample can be simulated. Source emission spectrum can be conditionally specified in the form of a distribution having a finite width, for example, the Gaussian distribution (8):

$${S}_{LS} ( λ ) = {e}^{- \frac{4 {( λ - {λ}_{0} )}^{2}}{{Δλ}^{2}}} \qquad Equation 8$$

where
S_{LS}(λ) is emission spectrum of light source (LS), arbitrary units,
Δλ is frequency band of light source (nm),
λo is central wavelength of light source (nm).

In this case, emission spectrum S′_{LS}(λ) of the light source, after passing through the sample, here, blood constituent, can be represented by equation 9:

$${S}_{LS}^{′} ( λ ) = {S}_{LS} ( λ ) * TtHb ( λ ) \qquad Equation 9$$

where S_{LS}(λ) is emission spectrum of light source, incident on the sample (arbitrary units),

TtHb(λ) is transmittance of blood constituent sample, here, total hemoglobin, at wavelength λ.

Disregarding spectral response curve of the photodetector, total intensity I_{PD} detected by photodetector (PD) is determined by equation 10:

$${I}_{PD} = {\sum }_{λ} {S}_{LS}^{′} ( λ ) \qquad Equation 10$$

where S′_{LS}(λ) is emission spectrum of light source after passing through the sample.

Equation 10 can be used to calculate total intensity of emission from a source with central wavelength λo, after passing through the sample. For example, calculation can be made for source LS1 with center wavelength λ₀^{LS1} and for source LS2 with center wavelength λ₀^{LS2}. For spectrophotometric measurements, not absolute values of intensities at specific wavelengths are important, but the ratio of emission intensities measured at different wavelengths (11):

$$I\_LS1 / I\_LS2 = \frac{{I}_{PD}^{LS 1}}{{I}_{PD}^{LS 2}} \qquad Equation 11$$

I_LS1/LS2 is ratio of emission intensities (I) of light source 1(LS1) and light source 2(LS2),

$${I}^{\frac{LS 1}{PD}}$$

is total emission intensity from light source LS1, passed through the sample and measured by photodetector (PD) (arbitrary units).

$${I}^{\frac{LS 2}{PD}}$$

_{is total emission intensity from light source LS2, passed through} the sample and detected by photodetector (PD) (arbitrary units).

As an example for calculations, the inventors considered radiation at wavelengths of 670 nm and 940 nm, which are often used in pulse oximetry schemes. With accuracy to the calibration factor, the ratio of intensities at selected wavelengths gives a blood oxygenation estimate. Methods for calculating the calibration factor to estimate the degree of oxygenation are disclosed in the publication: Toshiyo Tamura, Current progress of photoplethysmography and SPO₂ for health monitoring, Biomedical Engineering Letters (2019) 9:21-36, https://doi.org/10.1007/s13534-019-00097-w.

As already described earlier, when broadband radiation passes through analyzed volume, in particular, biological tissue, radiation at different wavelengths is absorbed differently (see FIG. 2C). Some wavelengths are absorbed to a greater extent, while other wavelengths are absorbed to a lesser extent. As a result, the initial source emission spectrum is distorted after passing through the studied volume.

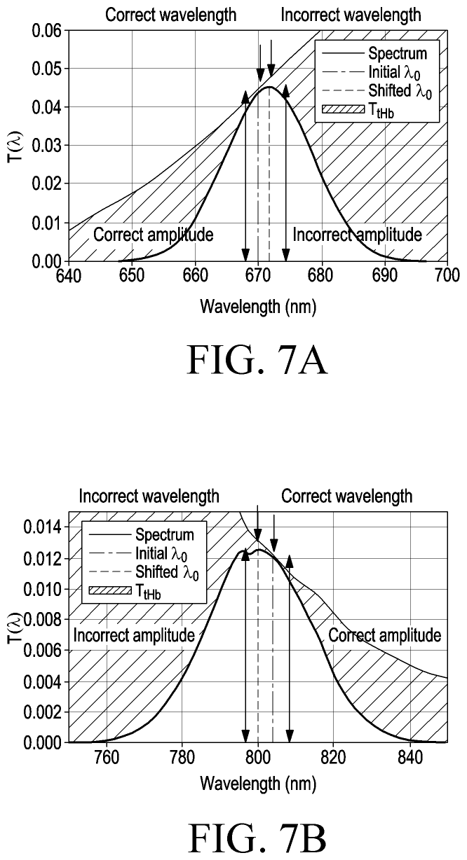

FIG. 7A schematically shows variations in emission spectrum of a broadband light source (bold line) at wavelength 670 nm when passing through the analyzed volume, which is defined by transmission spectrum ((T(o)) according to an embodiment of the disclosure.

FIG. 7B schematically shows variations in emission spectrum of a broadband light source (bold line) at wavelength 805 nm when passing through the analyzed volume, which is defined by transmission spectrum ((T(o)) according to an embodiment of the disclosure.

Referring to FIGS. 7A and 7B, bold line schematically shows variations in emission spectra of a broadband light source after passing through analyzed volume (x-axis represents wavelengths (nm)); solid filled area stands for transmission spectrum (T(X)) obtained from the mathematical model described above based on fulfillment of relations (8)-(11). Plots 7a and 7b show variations in broadband emission spectrum (bold black line) after passing through the analyzed volume; two spectra at different wavelengths 670 nm and 805 nm, respectively, are shown as an example.

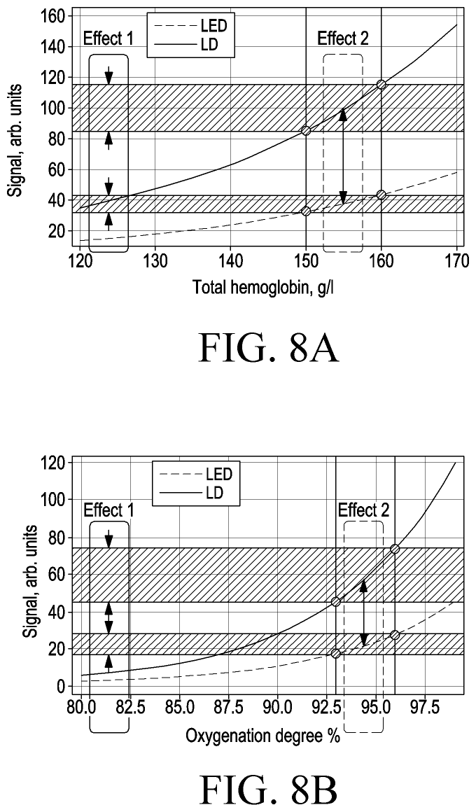

Spectrum shape has changed insignificantly, but spectral composition of the emission has undergone significant changes. Central wavelength is biased. Plots 7a and 7b show position of correct (initial) central wavelength of source emission (solid line) and central wavelength of emission (incorrect) after passing through the object studied (dashed line). Intensities of correct and incorrect wavelengths are also different. For spectral measurements, it is important to accurately determine intensity of emission that has passed through the object under study at a specific wavelength. In this example, as a result of using a broadband light source, an incorrect (overestimated) intensity value is measured, which corresponds to shifted (incorrect) wavelength. The effects illustrated in FIGS. 7A and 7B, lead, as a consequence, to incorrect results described in detail with reference to FIGS. 8A and 8B.

Comparative analysis was carried out between the measurement method and device based on a narrowband coherent light source in accordance with the disclosure and conventional spectral measurement means based on broadband measurement sources.

FIGS. 8A and 8B show plots of results of evaluating or determining parameters of blood constituents, in this case the concentration of total hemoglobin and the level of oxygenation, depending on the use of broadband or narrowband light source.

The inventors calculated the ratio of emission intensities for broadband light source, in this case a light-emitting diode (LED), and narrowband coherent light source, in this case laser diode (LD), for different concentrations of total hemoglobin (tHb) and level of blood oxygen saturation (oxygenation degree) (SpO₂).

As commonly known, to determine a blood parameter, for example, oxygenation degree, the ratio of intensities of light that has passed through the analyzed tissue volume is measured at different wavelengths, i.e. the ratio of transmission (or absorption) of the tissue at these wavelengths. Thus, this parameter (the ratio of intensities of light passed through the tissue at different wavelengths) and the response of this parameter to any changes in the composition or the state of the analyzed tissue volume is one of the basic criteria defining the effectiveness of measurement device, such as accuracy, sensitivity, dynamic range, etc.

As an embodiment of the disclosure, based on a narrowband coherent light source, the inventors measured total emission intensity I_{PD}^{RED} at central wavelength of 670 nm (red band of the emission spectrum), passed through the sample and detected at photodetector (PD), and total emission intensity I_{PD}^{IR2} at central wavelength of 940 nm (infrared band of the emission spectrum), passed through the sample and detected at photodetector (PD), and calculated, based on the measured total intensities, the ratio of said emission intensities (hereinafter signal) I_RED/IR2 at 670 nm and 940 nm, respectively, for various concentrations of total hemoglobin (tHb) and oxygenation level (SpO₂). For comparison, similar calculations were also made for broadband LED light source. Central wavelengths of the broadband light source were chosen to be equal to central wavelengths of the narrowband light source: 670 nm and 940 nm, respectively.

FIG. 8A shows plots of signal curves (arbitrary units) for a broadband light source (LED) represented by dotted line and a narrow band light source (laser diode, LD) represented by solid line as a function of total hemoglobin concentration (g/L) according to an embodiment of the disclosure.

FIG. 8B shows plots of signal curves (arbitrary units) for broadband light source (LED) represented by dotted line and a narrowband light source (laser diode, LD) represented by solid line as a function of oxygenation level (%) according to an embodiment of the disclosure.

As can be seen in plots of FIGS. 8A and 8B, when the total hemoglobin concentration of the oxygenation degree change by the same value, the signal corresponding to narrowband light source has more pronounced feedback (Effect 1 in FIGS. 8A and 8B), i.e. sensitivity to variations in the measured parameter when a narrowband light source is used is 2-3 times higher than the sensitivity to measurements when a broadband light source is used.

Furthermore, as can be seen in FIGS. 8A and 8B, the curve of broadband source signal versus total hemoglobin concentration and blood oxygen saturation degree is substantially biased relative to similar curve for narrowband source. Moreover, using a narrowband light source, transmission (or absorption) of the analyzed tissue volume is measured at precisely set target wavelengths, i.e. 670 nm and 940 nm, respectively.

Furthermore, using a broadband light source, the total (or average) transmission (or absorption) of the analyzed tissue volume is measured in a wide wavelength range corresponding to the source radiation band including, inter alia, target wavelengths, i.e. 670 nm and 940 nm, respectively. Transmission and/or absorption of the analyzed tissue volume at the other wavelengths included in the wavelength range emitted by the broadband source may differ substantially from the transmission (absorption) at the target wavelengths.

Averaging, or summing, over the entire range of wavelengths emitted by a broadband source leads to error in measurements of transmission and/or absorption at target wavelengths and, consequently, to error in estimate of concentrations of blood constituents. Differences between the two curves reveal that values of total hemoglobin concentration and blood oxygen saturation measured by a device based on broadband sources differ substantially from true values measured exactly at the target wavelengths using narrowband sources. Therefore, as follows from FIGS. 8A and 8B, at the same hemoglobin concentrations and the same oxygenation degree, the accuracy of measurements is higher for the narrowband light source compared to the wideband light source (Effect 2 in FIGS. 8A and 8B).

Furthermore, target components are selected in the disclosure by their dynamic properties (Key Point 2).

### Key Point 2 of the Disclosure

Key Point 2 is based on coherent detection of interferometric signal from blood constituents.

For describing dynamic properties of blood constituents, it is worthwhile to explain processes that occur when these components are radiated with coherent light at certain wavelengths, conditionally, e.g. wavelengths λ₁ and λ₂ can be taken.

Target components (K-1, K-2) of blood and non-target components (K-A, K-B, K-C(not shown)) blood and tissues when exposed to radiation from different light sources, absorb and/or scatter, at least partially, light at a certain wavelength (see FIGS. 4A and 4B). For example, target component K-1 and non-target component K-A absorb radiation at the same wavelength, e.g. λ₂, but target component K-1 is moving and non-target component K-A is static, and component K-2 and non-target component K-B absorb radiation at wavelength λ₁, but target component K-2 is moving and non-target component B is static.

When coherent light is scattered by a moving particle (e.g. blood cell), light parameters (frequency, wavelength) change, in accordance with the Doppler effect, by a small amount. Magnitude of this change is proportional to velocity of particle. When coherent radiation is scattered by a static (not moving) particle (for example, other components of biological tissue), light parameters (frequency, wavelength) do not change. When coherent radiation, which has experienced scattering by a moving particle, and reference coherent radiation are mixed, an oscillation signal (interferometric signal) appears at the photodetector aperture, which fluctuates at a difference frequency (period). Reference radiation can be both the radiation scattered by a moving particle or the radiation scattered by a static particle. The difference frequency (period) of the interference signal is proportional to the difference between parameters of two interfering parts of radiation (frequency, wavelength).

Performance of Key Point 2 implies the following possible initial conditions for target and non-target blood constituents(components):
target components K-1 and K-2 move and respective signals S_{λ1} and S_{λ2} registered at the photodetector from these components K-1 and K-2, are rapidly fluctuating,
non-target components K-A and K-B do not move and respective signals S_{λ1} and S_{λ2} registered at the photodetector from components K-A and K-B change slowly over time or remain constant.

Thus, from the point of view of coherent detection, signal incident on photodetector (light sensor) can be divided into a constant component and a fluctuating component, which constitute total signal at the photodetector from laser diode 1 (LD₁), equal to the sum of two components:

$${S}_{{λ}_{1}} = {S}_{{λ}_{1}}^{-} + {S}_{{λ}_{1}}^{-} \qquad Equation 1$$

where
S_{λ1} is total signal registered at photodetector, S_{λ}_{₁} is a fluctuating signal (component) registered at photodetector, and S_{λ}_{₁} is constant signal (component) registered at photodetector.

Total signal at the photodetector from laser diode 2 (LD₂) is equal to the sum of two components:

$${S}_{λ2} = {S}_{{λ}_{2}}^{-} + {S}_{{λ}_{2}}^{-} \qquad Equation 2$$

where S_{λ2} is total signal registered at photodetector, S is a fluctuating signal (component) registered at photodetector, and S_{λ}_{₂} is constant signal (component) registered at photodetector.

Thus, fluctuating signal S_{λ}_{₁} registered at photodetector, which is obtained during operation of laser diode 1 (LD₁), carries information only about target component K-1, and fluctuating signal S_{λ}_{₂} registered at photodetector, which is obtained during operation of laser diode 2 (LD₂), carries information about target component K-2.

Consequently, when total signal is registered at photodetector, constant component of the signal can be removed from it, and thereby fluctuating signal component can be obtained, so the target component can be determined by its dynamic properties, in this case, movement.

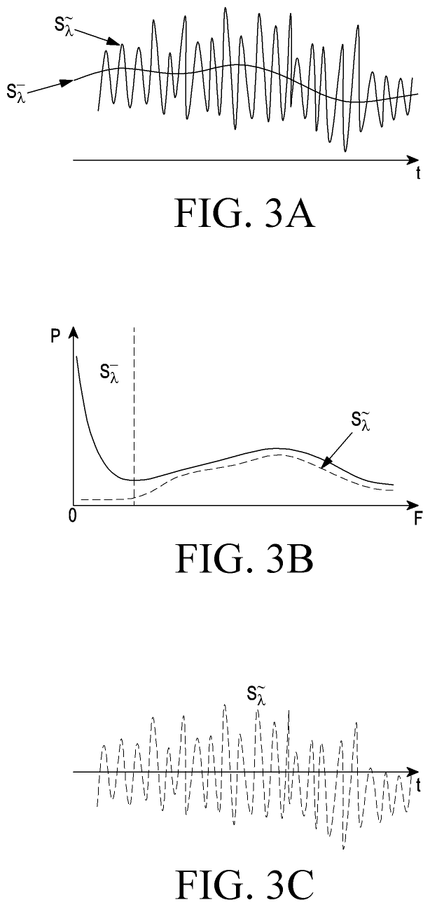

Further, with reference to plots in FIGS. 3A, 3B and 3C and FIGS. 4A, 4B, 4C, 4D, and 4F, a process of selecting target blood constituents will be described in more detail. Light at a wavelength λ₁, emitted by light source LD₁, is scattered at target and non-target blood constituents(components), information about which, when detected at PD photodetector, is contained in a conditionally constant or slowly changing and fluctuating signal components. Similar process occurs for light source LD₂. Signals from LD₁, LD₂ can be described by expressions (1) and (2) described above and defining total signals (S_{λ1} or S_{λ2}), each consisting of constant and fluctuating signal component.

FIG. 3A shows amplitudes of a constant (slowly changing) signal component and a fluctuating (rapidly changing) signal component according to an embodiment of the disclosure.

Total signal, conditionally S_{λ}, (1) is detected in a certain time period, and amplitudes of constant (S_{λ}) and fluctuating (S_{λ}) components of the signal are shown in FIG. 3A, where spectrum of high-frequency regions of the signal amplitude corresponds to fluctuating component of the signal from the moving component, spectrum of low-frequency component of the signal corresponds to slowly changing, i.e. substantially constant component of the signal from the non-moving component.

Next, (2) spectrum of said signal with given band corresponding to fluctuating component (s_{λ}) from the moving component and constant component (S_{λ}) from the static component, is selected.

FIG. 3B shows curves defining variation in signal power (P) as a function of signal frequency (F) for fluctuating component (S_{λ}) from moving component and constant component (S_{λ}) from static component, where solid line shows power of initial signal containing both slowly changing and rapidly changing component, and dotted line shows power of fluctuating component only according to an embodiment of the disclosure.

At the next step, by filtering and processing the resulting signal is restored, which contains only fluctuating component (S_{λ}). FIG. 3C shows amplitude of the resulting signal as a function of time for only fluctuating component (S_{λ}) corresponding to moving components according to an embodiment of the disclosure.

Next, processes will be explained that take place when the studied volume of subject's body is radiated with one or more light sources (LD₁, LD₂).

FIG. 4A is a schematic diagram illustrating a process of selecting components by dynamic properties, which occurs in the target zone radiated with coherent light source LD₁ at wavelength λ₁, where reference numerals 1 and 2 stand for light (radiation) beams interacting with moving blood particles (target components representing blood), and reference numeral 3 stands for light (radiation) beams interacting with non-moving particles (non-target component representing tissue or static tissue component) according to an embodiment of the disclosure. The light beams are light signals 1, 2 and 3, which are registered at photodetector (PD).

Radiating target (K-1), (K-2) and non-target (K-A), (K-B) components with coherent light causes interference of light waves with each other and with reference light, in this context, reference light means light scattered at target and non-target components, light reflected from surface of tissue (skin), light reflected from interfaces of any optical elements used in the device structure (lenses, mirrors, windows, films, coatings, etc.).

Furthermore, three options for interference of light signals can be considered:

FIG. 4C is the amplitude of signal fluctuating as a result of interference of light scattered on moving component K-1 and reference light according to an embodiment of the disclosure.

1) Interference of light from target moving component K-1 and reference light and from target moving component K-2 (see FIG. 4B) and reference light, resulting in formation of high-frequency, strong (high power) fluctuating signal (signal frequency band 2), see FIG. 4C.

FIG. 4D is the amplitude of signal fluctuating as a result of mutual interference of light scattered on target moving component K-1 and target moving component K-2 according to an embodiment of the disclosure.

2) Mutual interference of radiation (light beams 1, 2) from target moving component K-1 and from target moving component K-2 (signal frequency band 1), resulting in formation of low-frequency weak (low power) signal, see FIG. 4D.

Signal (light beam 3) from non-target non-moving component K-A (and/or component K-B) is slowly varying signal (signal frequency band 0) as shown in FIG. 4E. Here, signal frequency bands 1 and 2 define S_{λ}_{₁} (fluctuating signal), and signal frequency band 0 define S_{λ}, (constant signal).

Photodetector (PD) registers light signals 1, 2 and 3, as described by the above relationship

S_{λ}_{₁}=S_{λ}_{₁}+S_{λ}_{₁} (1).

FIG. 4E is the amplitude of non-fluctuating (slowly varying) signal from static component K-A according to an embodiment of the disclosure.

FIG. 4F is a curve defining power of signal registered at photodetector (PD) as a function of frequency, in signal frequency band 0 corresponding to signal in FIG. 4E, in signal frequency band 1 corresponding to signal in FIG. 4D and signal frequency band 2 corresponding to signal in FIG. 4C according to an embodiment of the disclosure.

Both fluctuating signals (S_{λ}_{₁}) in frequency band 1 and in frequency band 2, and in both frequency bands 1 and 2 were measured at the photodetector (PD) (see FIG. 4F), which improved accuracy of absorption measurements of only blood constituents, excluding other components of the subject's target area.

In addition, the ability of excluding (filtering out) constant signal S_{λ}_{₁}, (frequency band 0 in FIG. 4F) from total signal S_{λ1}, defining motion artefacts and/or noise, ensures high resistance to displacements of measurement device and measured object relative to each other.

It should be noted that filtering out of detected interference signals, particularly, constant signal S_{λ}_{₁}, from total signal S_{λ1}, as mentioned above, is performed in given frequency band, which contains frequencies of interferometric oscillations defining dynamics of blood constituents, using an analog or digital filter, e.g. a high-pass filter or band-pass filter. At the step of filtering out detected interference signals in given frequency band, the given frequency band F can be in the range from Fₘᵢₙ=500 Hz to F=10 kHz, and correspond to higher frequencies of interferometric oscillations compared to the other detected interference signals registered at the at least one photodetector, and the given frequency band F can be in the range from Fₘᵢₙ=50 Hz to Fₘₐₓ=500 Hz, and correspond to lower frequencies of interferometric oscillations compared to the other detected interference signals registered at the least one photodetector.

However, the aforementioned ranges are not limited to the values presented, and are given in the present description only as an example.

Next, filtered signals are sampled, using an ADC, at a frequency corresponding to respective given bandwidth of the filtered signal, to form discrete filtered signals. AD conversion of the filtered signal is carried out at sampling frequency F_{D} satisfying the Nyquist criterion for given frequency band F: F_{D};>2·Fₘₐₓ and at sampling frequency F_{D} below the threshold corresponding to the Nyquist criterion for given frequency band, F_{D}<2·Fₘₐₓ, so that after AD conversion, the filtered high-frequency signal is transferred to the low-frequency domain: F<F_{D}/2.

Further, spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) defining spectral and dynamic properties of blood constituents(components), are selected from the discrete filtered signals,

where λ₁, λ₂, λ₃, . . . λₙ is wavelength index,

t₁, . . . tₖ is time feature index, with spectral-time feature v_{λ}_{ₙ}^{tk} corresponding to time feature tₖ measured at the wavelength λₙ of said discrete filtered signals.

It should be noted that the set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals for each of at least two wavelengths is at least one of: scattered emission intensity, total signal power, power in given signal bandwidth, signal spectrum moments, characteristic (dominant) signal frequencies, and combinations of the spectral-time features, absolute values of said spectral-time features, relative values of said spectral-time features, linear combinations of said spectral-time features at different wavelengths, non-linear combinations of said spectral-time features at different wavelengths.

Based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals, concentration c1 . . . cm of blood constituents is calculated.

Blood constituents are determined by following relationships (3)

$${c}_{1} \approx {f}_{1} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{2} \approx {f}_{2} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) … \\ {c}_{m} \approx {f}_{m} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \qquad Equation 3$$

where c₁ . . . cₘ are concentrations of blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}),
f₁, f₂, fₘ are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals and concentrations of blood constituents being determined.

The process that occurs in the analyzed volume of tissue during irradiation has been described in detail above with references to FIGS. 4A, 4B, 4C, 4D, 4E, and 4F.

In one preferred embodiment, electrical signal registered at photodetector PD is filtered by an analog or digital high-pass filter, thereby retaining for further processing the interferometric signal located in signal frequency band 2 (FIG. 4F) from about 500 Hz to 10 kHz, and corresponding to higher frequencies of interferometric oscillations compared to the other detected interference signals registered at the photodetector. The aforementioned range is not limited to the values presented, but is given in the description only as an example.

As previously explained, interference of light from target moving component K-1 and reference light, and light from target moving component K-2 and reference light, results in formation of high-frequency, strong (high power) fluctuating signal (frequency band 2) (see FIGS. 4C and 4D).

As already described above, this particular high-frequency fluctuating signal, defining moving and/or pulsatile blood components(constituents), is subject to detection and further AD conversion and processing.

FIG. 5 is a schematic diagram illustrating a process occurring in analyzed tissue volume when radiated with multiple coherent light sources according to an embodiment of the disclosure.

The number of narrowband coherent light sources in the inventive device is not limited to two, there may be a plurality of them, i.e. two or more: LD₁, LD₂ . . . LDₙ with wavelengths λ₁, λ₂, . . . λₙ, respectively (cM. FIG. 5).

Moreover, increasing the number of light sources can enhance the measurement accuracy and sensitivity to SpO₂ measurements and to all blood hemoglobin measurements in the analyzed tissue volume. In addition, it widens opportunities of studying additional parameters of the analyzed tissue volume, in addition to the aforementioned oxygenated oxygen SpO₂ and level of total hemoglobin (Hb), and only level of total hemoglobin (Hb), these parameters can include carboxyhemoglobin (COHb), methemoglobin (MetHb), etc., substantially expanding the range of parameters of blood constituents(components) measured in real time for the user.

As explained earlier, one of the main points of data acquisition from signals detected at photodetector PD is filtering the signal in a certain frequency band.

In one embodiment of the disclosure, signal is filtered using an analog or digital broadband filter to extract interferometric signal from frequency band 1 for further processing (FIG. 4F).

As described earlier, as a result of mutual interference of light beams 1, 2 from target moving component K-1 and from target moving component K-2, the frequency band 1 is approximately from 50 Hz to 500 Hz) and corresponds to lower frequency interferometric oscillations compared to the other detected interference signals registered at the photodetector. However, the aforementioned range is not limited to the values presented and is given in the present description only as an example. Thus, a low-frequency low power signal is obtained, as shown in FIGS. 4D and 4F.

The low-frequency signal also defines pulsatile blood constituents and is subject to further processing. To do this, the signal is pre-processed with a band pass filter to select only those signal components that correspond to frequency band 1 of the signal. The signal is then sampled at a sampling frequency equal to or greater than the frequency sufficient to satisfy the Nyquist criterion for the frequency band 1 of the signal detailed earlier.

This embodiment facilitates the use of the present measurement method in conjunction with conventional microcircuits and controllers for working with PPG signals without additional requirements to modify existing processing and measurement means.

Next, the correlation between measured values of intensity of light passed through the sample and spectral and time features selected from the signal, and the concentration of desired component should be determined. To do this, a function or operation should be found, which describes this correlation. For this, concentrations of blood constituents for different populations of subjects, obtained e.g. in the course of laboratory studies, are used.

Next, at least one backscattered coherent light feedback from blood constituents and dynamic and static tissue components, which is at least one interferometric signal from each of at least two narrowband coherent light sources, is measured, followed by filtering the detected signal, AD conversion (sampling) of the filtered signal, selecting spectral and time features of the signal for the subject with known concentration of desired blood constituents. Next, the form of functional relationships ƒ₁, ƒ₂, ƒₘ and the values of coefficients included in them and associating the set of calculated spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals and known concentrations of components c₁ . . . cₘ is determined.

The aforementioned functional relationships ƒ₁, ƒ₂, ƒₘ can be found explicitly. For example, but not only, the function can be a regression model, which includes, as variables, measured values of transmitted light intensity, their ratios, or results of other mathematical transformations of the intensity values, while coefficients of the regression model are in the process of calibration. Otherwise, this function can be specified implicitly, e.g. in the form of a neural network receiving at input measured values of transmitted light intensity, their ratios, or results of other mathematical transformations of the intensity values. Coefficients of internal layers of the neural network are also in the process of calibration or training. After determining the type and parameters of the function that correlates intensity values of light transmitted through the sample and concentration of desired component in the calibration process, this function can be used to determine unknown concentration of the desired component. Methods for signal processing, in this case, methods for determining functions that define the correlation between measured values of intensity of light transmitted through sample (transmission and/or absorption spectrum) and concentration of desired component by various computer simulation methods are widely known, for example, from the publication: Ian Goodfellow, Yoshua Bengio, and Aaron Courville. 2016. Deep Learning. The MIT Press Maria Deprez, Emma Robinson, Machine Learning for Biomedical Applications, 2022, Elsevier.

Measurement of intensities I_{λ1}, I_{λ2}, . . . I_{λn} of light passed through the analyzed volume at least at two wavelengths, e.g. λ₁, λ₂, . . . λₙ. Light intensity is measured using at least two light sources emitting at wavelengths λ₁, λ₂, . . . λₙ, respectively, and at least one light sensor (photodetector), arranged such that light from the light sources passes through the analyzed volume of tissue and/or blood and is incident on the light-sensitive surface of the photodetector. Electrical signal generated by the photodetector, which is proportional to the intensity of light passed through the analyzed tissue volume, is amplified, optionally filtered and further processed in digital form.

2) Calculation of functions from at least two measured light intensities ƒ(I_{λ1}, I_{λ2}, . . . I_{λn}) or based on other parameters (e.g. ratio of intensity of light incident on the analyzed blood constituent and intensity of light that passed through the analyzed blood constituent), implicitly correlating light intensity and concentration of the analyzed blood constituent, using regression models, for example, at least one of the methods: regression method, including method of linear regression, logistic regression, method of successive approximations, differential method, including gradient method, including gradient descent, stochastic gradient descent, and modifications of these methods, as well as machine learning models, is disclosed, for example, in publications: Ian Goodfellow, Yoshua Bengio, and Aaron Courville. 2016, Deep Learning. The MIT Press

Maria Deprez, Emma Robinson, Machine Learning for Biomedical Applications, 2022, Elsevier.

3) Estimation of concentration of a blood constituent of interest, in this non-limiting example of oxygenated hemoglobin, based on the obtained extinction coefficients, absorption coefficients, and transmittance:

O₂Hb≈ƒ(I_{λ1},I_{λ2}, . . . I_{λn}).

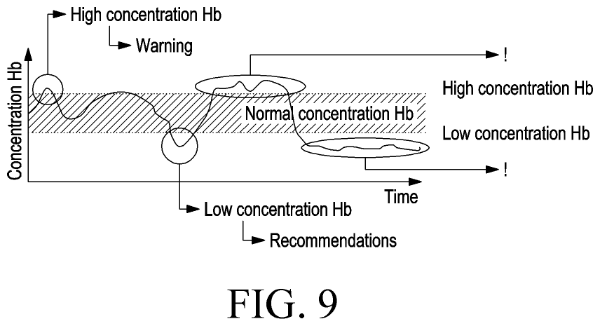

FIG. 9 is a schematic diagram of noninvasive monitoring of hemoglobin concentration in blood or other blood constituents versus time according to an embodiment of the disclosure.

Here, two observation options are considered: 1) long-term, where the observation period is from several hours to several days or even up to several months, and 2) short-term, where the observation period is from several minutes to several hours.

When monitoring concentration of hemoglobin, the following situations are possible. If a high concentration of hemoglobin (Hb) is determined, the device user is warned about the result obtained, if a low concentration of hemoglobin is determined, the user is informed about the result obtained and a preliminary diagnosis, for example, anemia, with general recommendations displayed on the screen: change the diet, increase activity, walk in fresh air, etc.

Moreover, short-term deviations of hemoglobin concentration from the norm are not critical and can be corrected by the device user using the displayed recommendations, while on the contrary, long-term deviations of hemoglobin concentration from the norm can mean serious health problems, and recommendation on mandatory visit to a specialist is displayed. In addition, data obtained from the results of short-term or long-term blood concentration monitoring can be sent to a specialist directly from the measurement device.

Advantages of such monitoring include rather low cost of the study, provision of long-term continuous monitoring of blood parameters, reliable measurement data and more accurate diagnosis of a person based on the data obtained.

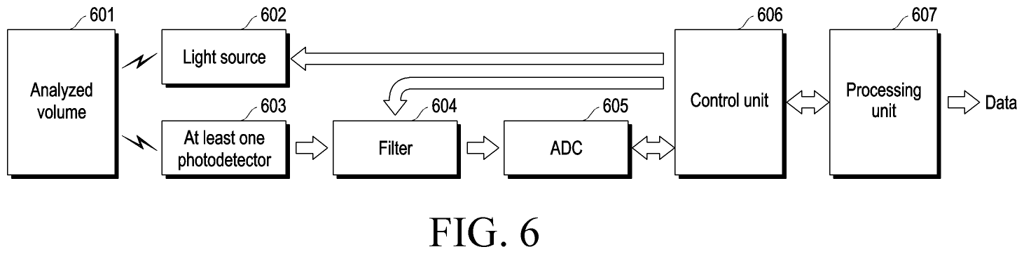

FIG. 6 is a structural diagram of a wearable device for noninvasive measurements of blood constituents according to an embodiment of the disclosure.

Further, referring to FIG. 6, a structural diagram of a wearable device for noninvasive measurements of blood constituents will be disclosed. An analyzed volume 601 is an area of biological tissue of a subject (human or animal to be examined), representing dynamic components, e.g. blood, erythrocytes, and static components such as tissue, chromophores and any non-moving components of the analyzed volume. A light source 602 is at least two narrowband coherent light sources, each having respective wavelength and configured to radiate the analyzed subject's body area containing blood constituents and dynamic and static tissue components.

Each of the at least two narrowband light sources is a laser diode (LD) and provides light at different wavelengths in the visible and near infrared regions of the spectrum, but these ranges do not limit possible emission spectra, and are given only as preferred examples.

At least one photodetector 603, includes a photocell, which is at least one of: a photodiode, a phototransistor, a photoresistor, or a charge-coupled device (CCD), or a complementary metal-oxide-semiconductor (CMOS) structure device, or another photosensitive element, and a photocurrent-to-voltage converter. The photodetector 603 is configured to detect and amplify at least one backscattered coherent light feedback from dynamic blood constituents and static tissue components, representing at least one interferometric signal from each of the at least two narrowband coherent light sources.

A filter 604 is an analog or digital filter, and is configured to extract a signal from the detected interference signals in accordance with a given signal bandwidth at a given time.

An analog-to-digital converter (ADC) 605, or a sampler configured to AD conversion of the filtered signals at a frequency corresponding to a predetermined filtered signal bandwidth, forms discrete filtered signals.

A control unit 606 (e.g., at least one processor) may be configured to control at least two narrowband coherent light sources, at least one photodetector, and a filter, an analog-to-digital converter, and a processing unit 607 may be configured to process filtered interference signals obtained after sampling, and defining selected blood constituents, to obtain concentration values of selected dynamic blood constituents according to the method for noninvasive measurements of blood constituents. The control unit and the processing unit may be implemented or referred to as at least one processor.

Moreover, the processing unit 607 is connected to the control unit 606, and is configured to process the discrete filtered interference signals obtained after sampling, and perform the following steps by signal from the control unit:
selecting, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals, defining spectral and dynamic properties of blood constituents(components),
where λ₁, λ₂, λ₃, . . . λₙ is wavelength index,
t₁, . . . tₖ is time feature index, with spectral-time feature v_{λ}_{ₙ}^{tk} corresponding to time feature tₖ measured at wavelength λ₂,
calculating concentration of blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) in accordance with the expression:

$${c}_{1} \approx {f}_{1} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{2} \approx {f}_{2} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{m} \approx {f}_{m} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} )$$

where c₁ . . . cₘ are concentrations of blood constituents, which are defined by respective set of features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk})
ƒ₁, ƒ₂, ƒₘ are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) of signals and the concentrations of blood constituents being determined.

The device further comprises memory unit (not shown in FIG. 6) designed to store the parameters required for operation of the device elements (feed voltages and currents of the light source, ADC sampling rate, gain factors, calibration coefficient values for spectral and time features included in the model for calculating the concentration of blood constituents, etc.).

In addition, the device for noninvasive measurements of blood constituents is configured to be integrated in one of: a wearable personal health monitoring device, in particular, smart watch, stationary health monitoring diagnostic device, consumer devices and gadgets for personal healthcare.

The device and method for measuring blood constituents can be used for noninvasive, personal and/or on demand or request monitoring of the health status of a subject, in particular monitoring the measurement of concentration of various forms of hemoglobin Hb: oxygenated Hb, deoxygenated Hb, met-Hb, carboxy-Hb, and other blood properties, and can be used in wearable measuring devices, wrist watches, smart watches, stationary diagnostic devices, consumer devices and gadgets for personal healthcare.

The following systems for measuring blood parameters were analyzed:
device according to the disclosure,
Cercacor (U.S. Ser. No. 10/123,726B2),
mHematology, “Virtual Hyperspectral Imaging of Eyelids”-mHematology for blood hemoglobin analysis, Michelle A. Visbal-Onufrak, total 41 pages, publ. 12.04.2019, available at (http://dx.doi.org/10.2139/ssrn.3369797),
HemaApp, “Noninvasive blood screening of hemoglobin using smartphone cameras”, Doug Hawkins, et al., UbiComp '16: Proceedings of the 2016 ACM International Joint Conference on Pervasive and Ubiquitous Computing, September 2016, Pages 593-604, available at (http://dx.doi.org/10.1145/2971648.2971653),
Fingermale, “Smartphone app for noninvasive detection of anemia using only patient-sourced photos”, Rober G. Mannino et al., publ. 04.12.2018, available at (https://doi.org/10.1038/s41467-018-07262-2).

### Device According to the Disclosure:

1) no additional separate measurement means is required, can be integrated into existing wearable devices (mobile phone, watch, fitness bracelets, etc.),
2) high accuracy of measurements of blood parameters,
3) low power consumption,
4) continuous monitoring without user participation can be provided,
5) high resistance to parasitic movements.

### Cercacor:

1) requires additional separate measurement means (e.g. a finger clip),
2) sufficient accuracy in measuring blood parameters,
3) high power consumption,
4) no continuous monitoring is provided, each individual measurement requires direct participation of the user.
5) medium resistance to parasitic movements.
   mHematology:
1) no additional separate measuring means is required,
2) low accuracy of measurements of blood parameters,
3) low power consumption,
4) no continuous monitoring is provided,
5) ensures very low resistance to movement.

### Hema App:

1) requires additional separate measuring means and additional light sources,
2) low accuracy of measurements of blood parameters,
3) low power consumption,
4) continuous monitoring is not provided,
5) very low resistance to movement.

### Fingermale App:

1) no additional separate measuring means is required,
2) low accuracy of measurements of blood parameters,
3) low power consumption,
4) continuous monitoring is not provided,
5) very low resistance to movement.

Therefore, the comparative analysis of similar conventional systems on the market, carried out by the inventors, allows for the conclusion that the disclosure exhibits high accuracy of measurements of blood parameters, along with other advantages.

As would be apparent to a person in the art, various modifications may be made to the method in order to implement the disclosure as taught herein.

The drawings and the forgoing description give examples of embodiments. Those skilled in the art will appreciate that one or more of the described elements may well be combined into a single functional element. Alternatively, certain elements may be split into multiple functional elements. Elements from an embodiment may be added to another embodiment. For example, orders of processes described herein may be changed and are not necessarily limited to the manner described herein.

Moreover, the actions of any flow diagram need not be implemented in the order shown; nor do all of the acts necessarily need to be performed. Also, those acts that are not dependent on other acts may be performed in parallel with the other acts.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been illustrated and described with reference to various embodiments thereof, it will be understood by those skilled in the art that various that various changes in form and details may be made therein without departing from the spirit and scope of the disclosure as defined by the appended claims and their equivalents.

What is claimed is:

1. A method for measuring blood constituents by a device, the method comprising:
radiating an area of a subject's body, containing blood constituents and dynamic and static tissue components, with at least two light beams using at least two light sources;
detecting, using at least one photodetector, at least one backscattered light feedback from the blood constituents and dynamic and static tissue components, wherein the feedback represents at least one interferometric signal from each of the at least two light sources;
filtering, using a filter, the at least one interferometric signal in accordance with a signal frequency band which contains frequencies of interferometric oscillations;
performing analog-to-digital (AD) conversion of the filtered signals at a frequency corresponding to the signal frequency band of the filtered signal, to form discrete filtered signals;
obtaining, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), defining spectral and dynamic properties of the blood constituents,
where λ1, λ2, λ3, . . . , λn are wavelength indexes, and
where t1, . . . tk are time feature indexes, with spectral-time feature v_{λ}_{ₙ}^{tk} corresponding to time feature tk measured at wavelength λn; and
obtaining a concentration of the blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression:
$${c}_{1} \approx {f}_{1} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{2} \approx {f}_{2} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) … \\ {c}_{m} \approx {f}_{m} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} )$$
where c1, c2, . . . ,cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), and
where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

2. The method of claim 1, wherein the at least two light sources provide emission with different wavelengths in the visible and near infrared regions of spectrum.

3. The method of claim 1, wherein each of the at least two light sources is a laser diode (LD).

4. The method of claim 1, wherein the radiating of the area of the subject's body with at least two light sources is carried out by a signal from a control unit at predetermined time periods.

5. The method of claim 1, further comprising:
registering at the at least one photodetector from each of the at least two light sources, at least one interferometric signal Sa defining backscattered light feedback from blood constituents and dynamic and static tissue components, and defined by the relation:
$${S}_{{λ}_{1}} = {S}_{{λ}_{1}}^{-} + {S}_{{λ}_{1}}^{-} ,$$
where S_{λ} is total signal registered at the at least one photodetector,
where S_{λ} is constant or slowly changing signal specific to interference of light scattered by static tissue components,
where S_{λ} is fluctuating signal specific to the interference of light scattered by blood constituents and dynamic tissue components, and
where λ is one of wavelengths λ₁, λ₂, λ₃, . . . λₙ of each of said at least two said light sources.

6. The method of claim 1, wherein the filtering comprises removing constant signal S_{λ} defining static components of the tissue from at least one interferometric signal S_{λ}, and sampling fluctuating signal S_{λ} defining blood constituents and dynamic components of the tissue.

7. The method of claim 1, wherein a signal bandwidth is in a range from 500 Hz to 10 kHz, and the signal bandwidth corresponds to higher frequencies of the interferometric oscillations compared to interferometric signals registered at the at least one photodetector.

8. The method of claim 1, wherein a signal bandwidth is in a range from 50 Hz to 500 Hz, and the signal bandwidth corresponds to lower frequencies of interferometric oscillations compared to interferometric signals registered at the at least one photodetector.

9. The method of claim 7, wherein the AD conversion of the filtered signal is performed at sampling frequency F_{D} satisfying Nyquist criterion for the signal bandwidth F: F_{D}>2·Fₘₐₓ.

10. The method of claim 7, wherein the AD conversion of the filtered signal is performed at sampling frequency F_{D} below a threshold corresponding to Nyquist criterion for the signal bandwidth, F_{D}<2·Fₘₐₓ, so that after the AD conversion, the filtered signal is transferred to low-frequency domain: F<F_{D}/2.

11. The method of claim 1, wherein the set of spectral-time features of signals for each of at least two wavelengths includes at least one of:
scattered light intensity, total signal power, power in a given signal frequency band, signal spectrum moments, signal frequencies specific to interferometric oscillations frequencies defining dynamics of blood constituents, and combinations thereof, absolute values there, relative values thereof, linear combinations thereof at different wavelengths, non-linear combinations thereof at different wavelengths.

12. The method of claim 1, wherein the concentrations of blood constituents c₁, c₂, . . . , cₘ are predetermined values of concentrations of blood constituents for different populations of subjects or groups of subject populations, for which features of respective interferometric signals are predetermined, used in a calibration process.

13. The method of claim 1, wherein a type of the functional relationships, and values of coefficients included in the functional relationships are determined in a calibration process by at least one of: linear regression, logistic regression, successive approximations, gradient methods, gradient descent, stochastic gradient descent, and modifications thereof, based on previously measured data of blood constituents for different populations of subjects or groups of subject populations.

14. The method of claim 1, further comprising:
determining desired concentration of blood constituents in an analyzed object based on the measured values of spectral-time features, using the functional relationships determined in a calibration process.

15. A device for measuring blood constituents, the device comprising:
at least two light sources;
at least one photodetector;
a filter;
an analog-to-digital (AD) converter;
memory storing one or more computer programs; and
one or more processors communicatively coupled to the at least two light sources, the at least one photodetector, the filter, the AD converter, and the memory,
wherein the one or more computer programs include computer-executable instructions that, when executed by the one or more processors individually or collectively, cause the device to:
radiate an area of a subject's body, containing blood constituents and dynamic and static tissue components, with at least two light beams using at least two light sources;
detect, using the at least one photodetector, at least one backscattered light feedback from the blood constituents and dynamic and static tissue components, wherein the feedback represents at least one interferometric signal from each of the at least two light sources;
filter, using the filter, the at least one interferometric signal in accordance with a signal frequency band which contains frequencies of interferometric oscillations;
perform analog-to-digital (AD) conversion of the filtered signals at a frequency corresponding to the signal frequency band of the filtered signal, to form discrete filtered signals;
obtain, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), defining spectral and dynamic properties of the blood constituents,
where λ1, λ2, λ3, . . . , λn are wavelength indexes, and
where t1, . . . tk are time feature indexes, with spectral-time feature v_{λ}_{ₙ}^{tk} corresponding to time feature tk measured at wavelength λn; and
obtain a concentration of the blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression:
$${c}_{1} \approx {f}_{1} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{2} \approx {f}_{2} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) … \\ {c}_{m} \approx {f}_{m} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} )$$
where c1, c2, . . . ,cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), and
where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

16. The device of claim 15, wherein the at least two light sources provide emission with different wavelengths in the visible and near infrared regions of spectrum.

17. The device of claim 15, wherein each of the at least two light sources is a laser diode (LD).

18. The device of claim 15, wherein the radiating of the area of the subject's body with at least two light sources is carried out by a signal from one of the one or more processors at predetermined time periods.

19. One or more non-transitory computer-readable storage media storing one or more computer programs including computer-executable instructions that, when executed by one or more processors of an electronic device individually or collectively, cause the electronic device to perform operations, the operations comprising:
radiating an area of a subject's body, containing blood constituents and dynamic and static tissue components, with at least two light beams using at least two light sources;
detecting, using at least one photodetector, at least one backscattered light feedback from the blood constituents and dynamic and static tissue components, wherein the feedback represents at least one interferometric signal from each of the at least two light sources;
filtering, using a filter, the at least one interferometric signal in accordance with a signal frequency band which contains frequencies of interferometric oscillations;
performing analog-to-digital (AD) conversion of the filtered signals at a frequency corresponding to the signal frequency band of the filtered signal, to form discrete filtered signals;
obtaining, from the discrete filtered signals, a set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), defining spectral and dynamic properties of the blood constituents,
where λ1, λ2, λ3, . . . , λn are wavelength indexes, and
where t1, . . . tₖ are time feature indexes, with spectral-time feature v_{λ}_{ₙ}^{tk} corresponding to time feature tₖ measured at wavelength λn; and
obtaining a concentration of the blood constituents based on the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) according to following expression:
$${c}_{1} \approx {f}_{1} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) \\ {c}_{2} \approx {f}_{2} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} ) … \\ {c}_{m} \approx {f}_{m} ( {v}_{{λ}_{1}}^{{t}_{1}} , {v}_{{λ}_{2}}^{{t}_{1}} , … {v}_{{λ}_{n}}^{{t}_{1}} , … {v}_{{λ}_{1}}^{{t}_{k}} , … {v}_{{λ}_{n}}^{{t}_{k}} )$$
where c1, c2, . . . ,cm are concentrations of the blood constituents, which are defined by respective set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}), and where f1, f2, . . . , fm are functional relationships of the obtained set of spectral-time features (v_{λ}_{₁}^{t1}, v_{λ}_{₂}^{t1}, . . . v_{λ}^{t1}, . . . v_{λ}_{₁}^{tk}, . . . v_{λ}_{ₙ}^{tk}) and the concentrations of the blood constituents.

20. The one or more non-transitory computer-readable storage media of claim 19, the operations further comprising:
registering at the at least one photodetector from each of the at least two light sources, at least one interferometric signal Sa defining backscattered light feedback from blood constituents and dynamic and static tissue components, and defined by the relation:
$${S}_{{λ}_{1}} = {S}_{{λ}_{1}}^{-} + {S}_{{λ}_{1}}^{-} ,$$
where S_{λ} is total signal registered at the at least one photodetector, where S_{λ} is constant or slowly changing signal specific to interference of light scattered by static tissue components,
where S_{λ} is fluctuating signal specific to the interference of light scattered by blood constituents and dynamic tissue components, and
where λ is one of wavelengths λ₁, λ₂, λ₃, . . . λₙ of each of said at least two said light sources.
